(19) United States
(12) Patent Application Publication
(10) Pub. No.: US 2021/0059395 A1
(43) Pub. Date: Mar. 4, 2021

(54) LOCALIZATION SENSING METHOD FOR AN ORAL CARE DEVICE

(71) Applicant: KONINKLIJKE PHILIPS N.V., EINDHOVEN (NL)

(72) Inventors: GERBEN KOOIJMAN, LEENDE (NL); FELIPE MAIA MASCULO, EINDHOVEN (NL)

(21) Appl. No.: 16/977,147

(22) PCT Filed: Feb. 28, 2019
(86) PCT No.: PCT/EP2019/055063

§ 371 Date: Sep. 1, 2020

Related U.S. Application Data
(60) Provisional application No. 62/636,900, filed on Mar. 1, 2018.

(30) Foreign Application Priority Data
Feb. 7, 2019 (EP) 19155917.8

(51) Int. Cl. A46B 15/00; A46B 17/08; A46B 9/04; A61B 5/00; A61B 5/1178

(52) U.S. Cl. CPC A46B 15/0006; A46B 15/0008; A46B 17/08; A46B 2200/1066; A61B 5/0013; A61B 5/1178; A46B 9/04

(57) ABSTRACT

A method for monitoring the position of an oral care device in the mouth of a user, the method comprising emitting energy towards the user's face, receiving reflected energy from the user's face corresponding to the emitted energy, and determining the position of an oral care device in the mouth of the user using the received reflected energy and facial characteristics information of the user which relates to one or more facial features of the user.

### FIELD OF THE INVENTION

The present invention relates to a method and system for monitoring the position of an oral care device in the mouth of a user.

### BACKGROUND OF THE INVENTION

Determining position information of a handheld personal care device and its constituent parts relative to a user's body enables monitoring and coaching in personal cleaning or grooming regimens such as tooth brushing and interdental cleaning, face cleansing or shaving, and the like. For example, if the location of a head member of a personal care device is determined within the user's mouth, portions of a group of teeth, a specific tooth, or gum section may be identified so that the user may focus on those areas.

To facilitate proper cleaning techniques, some devices contain one or more sensors to detect location information of a handheld personal care device during a use session. Existing methods and devices detect orientation of a hand held personal care device using an inertial measurement unit, such as in power toothbrushes. However, the orientation data in the current devices does not uniquely identify all particular locations in the oral cavity. Thus, to locate the head member portion in a particular area of an oral cavity, the orientation data must be combined with guidance information. To enable this technology, the user must carry out a certain period of use session while following the guidance information to locate the head member portion within a certain segment of the mouth. Since this technology is based on orientation of the handheld personal care device relative to the world, movements associated with use of the device may not be differentiated from non-use movements (e.g., walking or turning head). As a result, the user is forced to limit his or her movement while operating the handheld personal care device if accurate location data is desired. Approaches which simply detect the presence or absence of skin in front of a sensor fail to account for user behaviour which may vary over time for an individual user and/or varies between users.

Accordingly, there is a need in the art for improved systems and methods for tracking the location of an oral care device within the mouth of the user.

### SUMMARY

It is desirable to provide a more robust method to determine the location of an oral cleaning device in a person's mouth. To better address this concern, according to an embodiment of a first aspect there is provided a method for monitoring the position of an oral care device in the mouth of a user, the method comprising emitting energy towards the user's face, receiving reflected energy from the user's face corresponding to the emitted energy, and determining the position of an oral care device in the mouth of the user using the received reflected energy and facial characteristics information of the user which relates to one or more facial features of the user.

The facial characteristics information may include information on one or more facial features of a user. The received reflected energy may be used to determine the position of an oral care device in the mouth of the user based on facial characteristics information. A portion of energy emitted towards a face of the user from the oral care device may be scattered or reflected by the face of the user (surface of the face). A portion of the reflected or scattered energy may be received by the oral care device. The distance of the oral care device from the face of the user may affect the amount of energy that will be detected. For example, the closer the emitting and detecting are conducted to the face of the user, the greater the amount of energy that will be detected. The geometry of the face of the user may relate to the reflected energy which is detected. For example, facial features which are angled with respect to the direction of the emitted energy may cause some of the reflected energy to be directed away from the oral care device, and less reflected energy may therefore be collected by the energy detector. The intensity of the reflected energy may therefore vary depending on the topology of the face of the user. The shape of facial features at which the energy is directed may therefore affect the signal produced due to the reflected energy. The detected energy may be processed to determine the shape of the feature from which the energy has been reflected, and may therefore determine which feature of the user's face the energy is emitted onto. The detected energy may vary when the same feature is detected due to the relative angle of the oral care device and the feature. The signal may be processed to determine the location of the facial feature with respect to the oral care device and the orientation of the oral care device with respect to the feature.

Facial characteristic information may be used to determine the location of the oral care device in the mouth of the user based on the facial feature which has been detected using the reflected energy. For example, the location of the mouth relative to the nose of the user of the oral care device may be used as the facial characteristics information. Energy reflected from the face of the user may indicate the location of the nose of the user. The facial characteristics information which may provide information on the location of the mouth of the user relative to the nose of the user may then be used to determine the position of the oral care device in the mouth of the user. The position of the oral care device in the mouth of the user may therefore be determined by reference to one or more facial features of the user. Any external facial features, such as size, shape and/or location of the eyes, nose, mouth may be used as the facial characteristics information. The reflected energy and facial characteristics information may indicate the orientation of the oral care device with respect to the user's face and/or the distance of the oral care device from the user's face and/or mouth, and/or the location of the oral care device within the mouth of the user. Thus, the facial characteristics may be used to determine where an oral care device is positioned with respect to the inside of the mouth of the user.

The position of the oral care device may include the location of the oral care device relative to the mouth of the user and/or or the face of the user. The position may indicate the location of a head of an oral care device within the mouth of the user, and/or in relation to the teeth and/or gums of the user. The position may include the angular position, or orientation, of the oral care device with respect to a vertical/horizontal direction or with respect to at least one facial feature of the user. The position may include the location and orientation of the oral care device in three dimensional space.

According to an embodiment of a further aspect, the facial characteristics information of the user may further comprise at least one of: data relating to one or more facial characteristics of the user, metadata related to the user, facial characteristics information derived from the received reflected energy.

The received reflected energy may be processed to provide information on the facial characteristics of the user, for example, the topology of the user's face. The received reflected energy may be processed to provide information on the location of the oral care device in relation to a facial feature of the user. During normal use of the oral care device, the received reflected energy may be collected so as to collate information on the facial characteristics of the user. A correlation between subsequently received reflected energy which indicates a facial feature and the location of other facial features of the user may be determined using the collated information. Thus, any subsequently received reflected energy may be used to determine the location of the oral care device with respect to the location of facial features of the user based on the previously received reflected energy. A representation of a portion of the face of the user, including facial features, may be produced by processing the collected reflected energy. Thus, a three dimensional (3D) map of the user may be created using the reflected energy. Information on the dimensions and relative location of facial features may be provided in the facial characteristics information. The received reflected energy may be collected in real-time, to build up information regarding facial characteristics of the user while the oral care device is in use. Once a sufficient amount of facial characteristics information has been collected, the collected reflected energy may be used to create a map or the like of the facial characteristics of the user.

Additionally or alternatively, prior to use the user may make a scanning motion of the oral care device a predetermined distance from the user's face while energy is emitted and received to and from the face of the user. This energy may be used to build a two-dimensional (2D) or 3D map or picture of the user's face.

Additionally or alternatively, data relating to one or more facial characteristics of the user, and/or metadata related to the user, may be used to provide the facial characteristics information.

The data and/or metadata may provide information on one or more facial features of the user, such as the size and/or shape and/or position of the nose, eyes, lips, teeth, jawline, cheekbones, facial hair, general face shape, hairline etc. The position of each feature may be determined with respect to the mouth of the user. Any feature of the face or head may be used. The position of an oral care device in the mouth of the user may be determined by using the position of a facial feature relative to the mouth of the user known from the facial characteristics information in conjunction with the facial feature detected using the received reflected energy. For example, the relationship (for example, the distance) between at least two facial features determined using the data and/or metadata may be used to determine the location of the oral care device with respect to one or more facial features of the user detected using the received reflected energy. Using the reflected energy in conjunction with the data and/or metadata and/or facial characteristics information derived from the received reflected energy means that a more accurate positioning of the oral care device in the mouth of the user may be determined. The metadata may be used to estimate, or improve the estimation, of facial features of the user using a predetermined correlation between the size/position of facial features and metadata.

According to an embodiment of a further aspect, the facial characteristics information of the user may be at least one of: obtained from an image of the user, input by the user, obtained by processing the received reflected energy.

An image of the user may be used to determine the facial characteristics of the user. For example, an image of the user may be processed to extract information on each facial feature of the user, or a selection of features, and determine their dimensions and/or locations. An image of the user may be a 2D image and/or a 3D image. The image may be input by the user, for example, by the user taking an image of their own face. The image may be taken by moving an imaging device at least part of the way around the head of the user to obtain a 3D image of the head and/or face of the user. The metadata may be input by the user. The metadata may be input manually or by voice command.

The image and/or input may be obtained before the oral care device is used. The same image and/or input may be used each time the method is performed. The image and/or input may be obtained when setting up the oral care device, and the determining may be performed by reference to the same image and/or data each time the method is performed thereafter.

The reflected energy may be used to generate an image, or map, of the user. As discussed above, a map or image of the facial characteristics of the user may be obtained while the oral care device is in use, by the collection and processing of the received reflected energy. The reflected energy may indicate the location of facial features relative to one another. The user may indicate via, for example, an app (application on a mobile phone, tablet, smart watch or the like), the location of the oral care device within their mouth while energy is emitted and received. The data and/or metadata may be input using an app.

According to a further aspect the metadata is based on information on at least one of: the user's weight, height, complexion, gender, age.

Using metadata such as the user's weight, height, complexion, gender and/or age allows the step of determining to more accurately correlate the received reflected energy with the information on the face of the user. The metadata may be used to predict the location of features of the user, for example, based on a predicted face shape of the user. The metadata may be used in conjunction with data and/or received reflected energy. The metadata may be used to improve the estimation of facial features of a user based on a determined correlation, for example, by correlating data on size and shape of facial features for particular groups of people sharing similar facial characteristics.

According to a further aspect, the position of the oral care device in the mouth of the user is determined using a mapping which indicates the position of the oral care device in the mouth of the user based on the received reflected energy and facial characteristics information of the user.

The mapping may be an algorithm which processes data to determine the position of the oral care device. The mapping may be a machine-learned algorithm, which may be taught using data input from multiple people in a controlled environment. For example, a known location of the oral care device in the mouth of the user may be correlated to the reflected energy gathered from multiple facial features of the multiple people. The mapping may be produced by compiling data on a plurality of users which it compares to the energy reflected from the user to determine the location of the oral care device in the mouth of the user. The mapping may provide information on the topology of a generic face. The mapping may provide information on the location of the oral care device relative to the face of a (generic) user based on reflected energy.

The mapping may be developed by collecting data on reflected energy which is received while each of the multiple people uses an oral care device. The reflected energy may be collected during a controlled session, in which the location of the oral care device in the mouth of a person is monitored while reflected energy is received. The reflected energy may then be processed to develop a mapping which correlates the received reflected energy from an average or generic person with respect to the location of the oral care device relative to their facial features. The mapping may define the relationship between facial characteristics of a generic person and received reflected energy. The mapping may be an image map.

The mapping may indicate the position of the oral care device in the mouth of the user based on the received reflected energy and/or on the data and/or metadata and/or facial characteristics information derived from the received reflected energy. The mapping may use facial characteristic information obtained from the received reflected energy and/or on the data and/or metadata to determine the position of particular facial features of the user relative to one another, or to the mouth of the user. The facial characteristics of the user may be used to calibrate the mapping so that the location of the facial features relative to the mouth of the user indicated by the mapping is specific to the user. For example, the mapping may be calibrated using an image of the user, where facial characteristics information including the position of facial features extracted from the image are used to calibrate the position of the equivalent features in the mapping. The distance of the user's mouth to the feature, and therefore the distance of the oral care device to the mouth of the user, may be determined. The mapping may be used to determine a topology map of the user's face based on the data and/or metadata and/or reflected energy.

Data relating to the reflected energy may be processed using the mapping. The received reflected energy may be input to the mapping to determine the location of the oral care device with respect to the user of the device. The mapping may indicate the position of the oral care device based on a combination of the reflected energy and the data and/or metadata and/or facial characteristics information derived from the received reflected energy. As discussed above, the data and/or metadata and/or facial characteristics information derived from the received reflected energy may be used to determine the dimensions and/or locations of facial characteristics of the user, for example, the topology of the user's face.

According to a further aspect the mapping is selected from a plurality of mappings, the selected mapping being a mapping which is determined to be the most relevant based on the facial characteristics information of the user.

Thus, the mapping may be selected from a plurality of stored mappings. Each of the plurality of stored mappings may relate to a person with different facial characteristics. The mapping most relevant to the facial characteristics of the user may be selected so that the position of the oral care device in the mouth of the user may be more accurately determined. A mapping which indicates the relationship between received reflected energy and a position of the oral care device based on facial features similar to that of the user of the oral care device may give a more accurate prediction of the position of an oral care device with respect to the user. Using such a mapping, the received reflected energy may correlate with a similar pattern of reflected energy which is received when a person with similar facial characteristics to the user uses the oral care device.

The mapping may be selected from the plurality of mappings based on the relevance of the facial characteristics information of the user to a group of people sharing similar facial characteristics. The mapping which has been developed based on facial characteristics of a group of people most similar to that of the user may be selected from the plurality of mappings. The facial characteristics information may be obtained from data of the user, metadata of the user and/or received reflected energy. Where an image of the user is used to obtain the facial characteristics information, the image (or features extracted from the image) may be compared to images (or features extracted from images) of the group of people who were used to create the mappings in order to determine which mapping should be selected.

The mapping may be selected based on one or more features of the user. For example, the mapping may be selected based on the nose of the user. The mapping which has been developed based on noses which are most similar to the nose of the user may be selected from the plurality of mappings. Alternatively, the mapping which has been developed based on a plurality of facial features which are similar to that of the user may be selected.

According to a further aspect each of the plurality of mappings may relate to a different group of people, each group sharing at least one of: certain facial characteristics, metadata. The certain facial characteristics and/or metadata of each group and the facial characteristics information of the user may be used to identify which group is most relevant to the facial characteristics information of the user. The mapping corresponding to the identified group may be selected.

Thus, each of the mappings may be based on a particular group of people who are considered to have shared facial characteristics. Each of the plurality of mappings may be developed by collecting information of the facial features of a group of people and collecting data on reflected energy which is received while each of the people uses an oral care device while the position of the oral care device in the mouth of the person is monitored.

Each group may comprise information on people with facial characteristics which are similar within a threshold. For example, the dimensions of a facial feature, such as width, length, location of a nose, for each of a plurality of people may be compared and the data on faces, or people, with a similar characteristic may be assigned to a particular group. Each group may have set ranges, for example a range of a dimension of a facial feature. One or more facial features of the user may be compared with one or more facial features of each group to see which of the ranges the dimensions of their facial features fall within, and the group may be selected based on this comparison.

For each group, a machine-learned algorithm may be provided as the mapping. Thus, each machine-learned algorithm (each of the plurality of mappings) may correspond to a different group of people. Each machine-learned algorithm may be trained in a controlled environment as described above, where each algorithm is trained using data input from multiple people within a particular group of people sharing similar facial characteristics. Thus, a different machine-learned algorithm may be provided for each group.

Thus, when the facial features of the user are compared to the facial features of the plurality of groups to determine which mapping should be selected based on which group has the most similar features, the selected mapping will indicate the position of the oral care device in the mouth of the user to a higher precision, as the selected mapping will have been trained using people with similar features to those of the user. Thus, the mapping will give a better indication of the location of the oral care device based on the features of the user.

According to a further aspect the mapping is adjusted based on the facial characteristics information.

The mapping may be adjusted based on the facial characteristics information of the user so that the map between the facial characteristics of the user and the location of the oral care device in the mouth of the user is improved. Thus, the location of the oral care device in the mouth of the user may be determined with a greater accuracy. For example, data relating to an image of the user may indicate information on each of the facial characteristics of the user. This information may be used to adapt the mapping, where, for example, the dimensions and location of the facial features of the user are correlated to the size and location of the facial features on which the mapping is based, and adjust the mapping based on the correlation so that received reflected light can be processed using the mapping to give a more precise indication of the location of the oral care device in the mouth of the user. Thus, when the adjusted mapping is used to process the received reflected energy, the determined position of the oral care device may more accurately reflect the actual position of the oral care device relative to the user. The mapping may be altered based on the received reflected energy, where the mapping is adapted as the user uses the oral care device, and information on the facial features of the user are indicated by processing the received reflected energy.

According to a further aspect the method may further comprise emitting setting energy towards the user's face; and receiving reflected setting energy from the user's face corresponding to the emitted energy; and determining the amount of the energy to be emitted towards the user's face in the step of emitting energy based on the reflected setting energy; or determining the amount of energy to be emitted towards the user's face in the step of emitting energy based on at least one of: data relating to one or more facial characteristics of the user, metadata related to the user.

The amount of energy to be emitted in the step of emitting energy may be set based on the characteristics of the user. For example, before the energy is emitted towards the user's face to determine facial characteristics, energy (“setting energy”) may be emitted towards the user's face to determine the amount of energy to be emitted subsequently. The setting energy may be emitted from a predefined distance from the user. The amount of reflected setting energy detected may indicate the adjustment required to be made to the energy to be emitted towards the user's face. Energy adjustment may be required depending on complexion of the user, for example, the skin tone of the user. A darker skin tone may require more energy to be emitted towards the user's face in order to receive sufficient reflected energy, whereas a lighter skin tone may require less energy to be emitted towards the user's face in order to receive sufficient reflected energy. The skin tone of the user may be determined by emitting a predefined amount of energy towards the face of the user from a predefined distance, and receiving reflected energy corresponding to the emitted energy. The amount of energy received may be compared to an average or predefined amount of expected reflected setting energy, which may indicate the amount that the emitted energy needs to be increased or decreased in order to obtain the desired amount of reflected energy.

The amount of energy to be emitted may additionally or alternatively be based on data and/or metadata of the user. For example, information on the skin tone of the user may be determined from an image of the user or input as metadata. The amount of energy to be emitted may then be based on a predetermined correlation between the skin tone of a person and the amount of energy that is required to be emitted in order to receive the required corresponding reflected energy.

Additionally or alternatively, the received reflected energy may be offset to adjust for differences between the received reflected energy and the desired reflected energy. For example, using the skin tone of the user determined in any of the ways described above, a signal produced as a result of the received reflected energy may be offset so as to compensate for a lower or higher signal received due to the skin tone of the user with respect to a predefined average.

According to a further aspect the energy may be at least one of: electromagnetic energy, acoustic energy.

The acoustic energy may be sonar, and acoustic frequencies used may include very low (infrasonic) to extremely high (ultrasonic) frequencies, or any combinations thereof. Reflections of sound pulses (echoes) may be used to indicate the distance, or dimensions, of an object.

According to a further aspect the electromagnetic energy may be at least one of: infra-red energy, radar energy.

According to a further aspect the receiving of reflected energy comprises a measurement based on a measurement of at least one of: capacitance, reflected intensity, reflected polarization.

According to a further aspect there may be provided a computer program product comprising code for causing a processor, when said code is executed on said processor, to execute the steps of any of the methods described above.

According to a further aspect there may be provided a computer program product comprising code for causing an oral care system to execute any of the methods described above.

According to a further aspect there may be provided a computing device comprising the computer program product as described above. The computing device may be a processor, or may comprise a processor.

According to a further aspect there may be provided an oral care system comprising an oral care device having an energy emitter and an energy detector; and a computing device comprising the computer program product, configured to receive and process signals from energy emitted and received by the oral care device.

The energy emitter/detector may perform the method step of emitting/detecting energy towards/from the user's face. The energy emitter may comprise an energy source. The oral care device may comprise one or more energy sources. The energy emitter may be directly integrated into a body portion of the device. The energy emitter and/or the energy detector may be arranged in a planar or curved surface of the oral care device. The energy emitter and/or the energy detector may be arranged so that they are outside the mouth during use. Energy emitters and energy detectors may be mounted together within a single package for ease of assembly of the oral care device or may be mounted separately with different positions and orientations within the oral care device. The energy emitter and energy detector may be arranged in close proximity to one another, or the energy emitter and energy detector may be arranged at a distance from one another. The energy emitter and/or the energy detector may be located anywhere within the device along a long axis of the device or around a circumference of the device.

The energy emitter may generate near infrared light energy using light emitting diodes and the energy detector may be configured to detect the wavelength of light emitted by the one or more energy emission sources. The energy detector may comprise photodetectors, for example, photodiodes or phototransistors, with spectral sensitivity which is consistent with detecting the wavelength of the light generated by the energy emitter.

The energy detector may be configured to generate sensor data, e.g. signals, based on received reflected energy and provide such sensor data to the computing device. The computing device may be formed of one or more modules and be configured to carry out the methods for monitoring the position of an oral care device in the mouth of the user as described herein. The computing device may comprise, for example, a processor and a memory and/or database. The processor may take any suitable form, including but not limited to a microcomputing device, multiple microcomputing devices, circuitry, a single processor, or plural processors. The memory or database may take any suitable form, including a non-volatile memory and/or RAM. The non-volatile memory may include read only memory (ROM), a hard disk drive (HDD), or a solid state drive (SSD). The memory may store, among other things, an operating system. The RAM is used by the processor for the temporary storage of data. An operating system may contain code which, when executed by computing device, controls operation of the hardware components of the oral care device. The computing device may transmit collected sensor data, and may be any module, device, or means capable of transmitting a wired or wireless signal, including but not limited to a Wi-Fi, Bluetooth, near field communication, and/or cellular module. The computing device may receive sensor data generated by the energy detector and assess and analyse that sensor data to determine the position of the oral care device in the mouth of the user.

According to a further aspect the oral care device may be a device chosen from a group of devices comprising: a toothbrush, a flossing device, an oral irrigator, a handle for receiving a care head for any of the foregoing devices, a care head for any of the foregoing devices. The computing device may be comprised in at least one of: a remote server, an interface device to provide user information about the use of the oral care device; wherein the interface device is chosen from a group of interface devices comprising: a smart phone, a tablet, the oral care device, the care head. The computing device may be provided in the oral care device.

### BRIEF DESCRIPTION OF THE DRAWINGS

Embodiments of the present disclosure may take form in various components and arrangements of components, and in various steps and arrangements of steps. Accordingly, the drawings are for purposes of illustrating the various embodiments and are not to be construed as limiting the embodiments. In the drawing figures, like reference numerals refer to like elements. In addition, it is to be noted that the figures may not be drawn to scale.

FIG. 1 is a diagram of a power toothbrush to which embodiments of aspects of the present invention may be applied;

FIG. 2 is a schematic diagram of an oral care system according to an aspect of an embodiment;

FIG. 3 is a diagram illustrating energy being emitted and detected to/from a face of the user according to an aspect of an embodiment;

FIG. 4 is a flow diagram illustrating a method of monitoring the position of an oral care device in the mouth of the user according to an aspect of an embodiment;

FIG. 5 is a diagram illustrating the relative position and dimensions of facial characteristics of the user according to an aspect of an embodiment;

FIG. 6 is a flow diagram illustrating a method of monitoring the position of an oral care device in the mouth of the user according to an aspect of an embodiment;

FIG. 7 is a flow diagram illustrating a method of a step of determining the position of an oral care device in the mouth of the user according to an aspect of an embodiment;

FIG. 8 is a flow diagram illustrating a method of monitoring the position of an oral care device in the mouth of the user according to an aspect of an embodiment;

FIG. 9 is a graph illustrating grouping of people based on their facial characteristics according to an aspect of an embodiment;

FIG. 10 is a flow chart illustrating a method of determining the amount of energy to be emitted towards the user's face according to an aspect of an embodiment; and

FIG. 11 is a flow chart illustrating a method of determining the amount of energy to be emitted towards the user's face according to an aspect of an embodiment.

### DETAILED DESCRIPTION

The embodiments of the present disclosure and the various features and advantageous details thereof are explained more fully with reference to the non-limiting examples that are described and/or illustrated in the drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the present disclosure. The examples used herein are intended merely to facilitate an understanding of ways in which the embodiments of the present may be practiced and to further enable those of skill in the art to practice the same. Accordingly, the examples herein should not be construed as limiting the scope of the embodiments of the present disclosure, which is defined solely by the appended claims and applicable law.

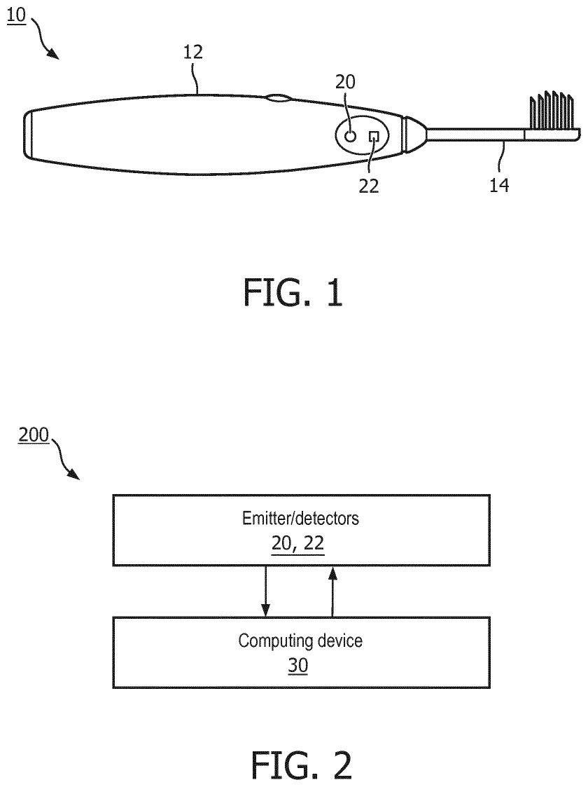

FIG. 1 shows an exemplary oral care device in which the teaching of the present disclosure may be implemented. The oral care device in FIG. 1 is in the form of an electric toothbrush (power toothbrush), but it will be appreciated that this is not limiting, and the teaching of the present disclosure may be implemented in other devices where location sensing is required. For example the teachings may be applied to personal care devices such as tongue cleaners, shavers, hair clippers or trimmers, hair removal devices, or skin care devices, and the position which is determined may be in relation to the surface of the face of the user, rather than the position within the mouth of the user.

Referring to FIG. 1 a handheld oral care device 10 is provided that includes a body portion 12 and a head member 14 removably or non-removably mounted on the body portion 12. The body portion 12 includes a housing, at least a portion of which is hollow, to contain components of the device, for example, a drive assembly/circuit, a computing device, and/or a power source (e.g., battery or power cord), not shown. The particular configuration and arrangement shown in FIG. 1 is by way of example only and does not limit the scope of the embodiments disclosed below.

Oral care device 10 includes one or more energy emitters 20 and one or more energy detectors 22 located in the handheld oral care device 10. The energy emitters and detectors 20, 22 may be directly integrated in the body portion 12 of the oral care device 10 (as shown in FIG. 1). Alternatively, the sources and detectors 20, 22 may be in a device attachment such as head member 14 or a module that may be attached to the device body portion 12. In this example, energy emitter 20 is configured to generate near infrared light energy using light emitting diodes and the energy detector 22 is configured to detect the wavelength of light emitted by the energy emitter 20.

Referring to FIG. 1, body portion 12 includes a long axis, a front side, a back side, a left side, and a right side. The front side is typically the side of the oral care device 10 that contains the operating components and actuators. Typically, operating components are components such as the bristles of a power toothbrush, the nozzle of a flossing device, the blade of a shaver, the brush head of a face cleansing device, etc. If the operating side is the front side of the body portion 12, the energy emitter 20 may be located on the right side of the body portion, opposite the left side, at its end proximate to the head member 14. However, the energy emitter 20 may be located anywhere within the device along the long axis or around a circumference of the oral care device 10. Similarly, the energy detector 22 may be located on the right side of the body portion, opposite the left side, at its end proximate to the head member 14. Although FIG. 1 depicts energy detector 22 located adjacent to the energy emitter 20, the energy detector 22 may be located anywhere within the device along the long axis or around a circumference of the device. Additional sensors may be included in the oral care device 10 shown in FIG. 1, including but not limited to a proximity sensor and other types of sensors, such as an accelerometer, a gyroscope, a magnetic sensor, a capacitive sensor, a camera, a photocell, a clock, a timer, any other types of sensors, or any combination of sensors, including, for example, an inertial measurement unit.

FIG. 2 shows a schematic representation of an example of an oral care system 200. The oral care system comprises an energy emitter 20 and an energy detector 22 and a computing device 30. The oral care system 200 may be implemented in one or more devices. For example, all the modules may be implemented in an oral care device. Alternatively, one or more of the modules or components may be implemented in a remote device, such as a smart phone, tablet, wearable device, computer, or other computing device. The computing device may communicate with a user interface via a connectivity module.

The oral care system 200 includes the computing device 30 having a processor and a memory (not shown), which may store an operating system as well as sensor data. System 200 also includes an energy emitter 20 and an energy detector 22 configured to generate and provide sensor data to computing device 30. The system 200 may include a connectivity module (not shown) which may be configured and/or programmed to transmit sensor data to a wireless transceiver. For example, the connectivity module may transmit sensor data via a Wi-Fi connection over the Internet or an Intranet to a dental professional, a database, or other location. Alternatively, the connectivity module may transmit sensor or feedback data via a Bluetooth or other wireless connection to a local device (e.g., a separate computing device), database, or other transceiver. For example, connectivity module allows the user to transmit sensor data to a separate database to be saved for long-term storage, to transmit sensor data for further analysis, to transmit user feedback to a separate user interface, or to share data with a dental professional, among other uses. The connectivity module may also be a transceiver that may receive user input information. Other communication and control signals described herein may be effectuated by a hard wire (non-wireless) connection, or by a combination of wireless and non-wireless connections. System 200 may also include any suitable power source. In embodiments, system 200 also includes the user interface which may be configured and/or programmed to transmit information to the user and/or receive information from the user. The user interface may be or may comprise a feedback module that provides feedback to the user via haptic signal, audio signal, visual signal, and/or any other type of signal.

Computing device 30 may receive the sensor data in real-time or periodically. For example, a constant stream of sensor data may be provided by the energy detector 22 to the computing device 30 for storage and/or analysis, or the energy detector 22 may temporarily store and aggregate or process data prior to sending it to computing device 30. Once received by computing device 30, the sensor data may be processed by a processor. The computing device 30 may relay information and/or receive information from the energy emitter and the energy detector 22.

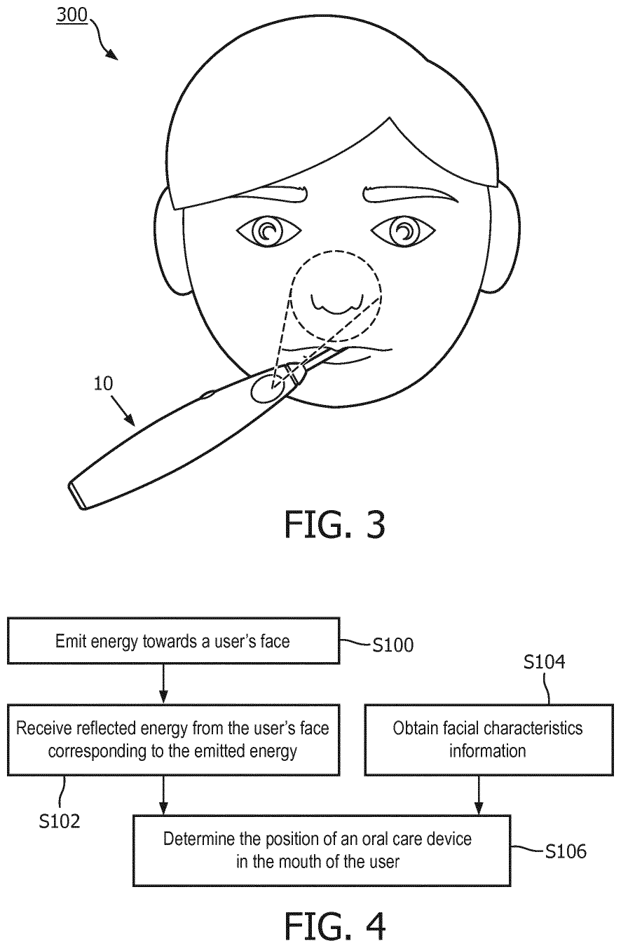

FIG. 3 shows an example of the oral care device 10 in use. In use, the oral care device 10 is inserted into the mouth of a user 300. Typically, the user 300 will move the oral care device around their mouth so that the teeth of the user 300 are brushed by the bristles of the head of the oral care device 10. In the example shown in FIG. 3, the energy emitter 20 provided on the oral care device 10 emits energy towards the face of the user 300. As is shown in this figure, the energy may be directed to a particular portion of the face of the user 300, in this case the nose of the user 300. Energy reflects off the nose of the user 300 and is detected by the energy detector 22 which is also provided on the oral care device 10. The detected energy which has been reflected from the face of the user indicates the dimensions of the portion of face onto which the emitted energy was directed and the distance of the feature to the oral care device 10, and the orientation of the oral care device 10 relative to the feature. In this case, the reflected energy will indicate the dimensions and position of the nose of the user 300.

The movement of the oral care device 10 relative to the face of the user 300 will cause energy to be directed onto different portions of the face of the user 300, for example, the eyes or mouth.

FIG. 4 shows a flow chart of an example of a method which may be performed for monitoring the position of the oral care device. In step S100, energy is emitted towards the user's face. In step S102, reflected energy is received from the user's face which corresponds to the emitted energy. For example, at least a portion of the energy which is emitted towards the face of the user will be reflected or scattered from the user's face. A portion of the reflected or scattered energy will be received. At step S104, facial characteristics information is obtained. For example, the facial characteristics information may be obtained from the reflected energy, or from data relating to one or more facial characteristics of the user, for example, an image of the user, or from metadata relating to the user, or a combination thereof. In step S106, the position of an oral care device in the mouth of the user is determined using the reflected energy and the obtained facial characteristics information.

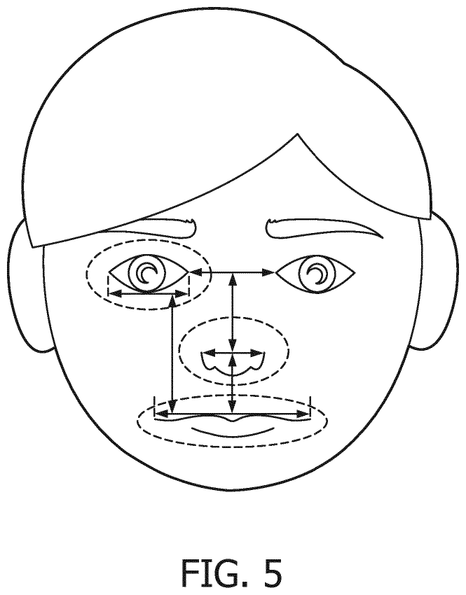

FIG. 5 shows an example of the relative position and dimensions of facial features of the user. FIG. 5 represents an image of the user from which facial characteristics are determined in step S104 of FIG. 4. In FIG. 5, the regions of the facial features of interest are indicated by dotted lines. In this case, the nose, mouth and eye of the user are indicated as regions of facial features of interest. The positions of the eye and nose relative to the mouth are determined as indicated by the arrows in FIG. 5. Facial characteristics such as the dimensions and location of each of the facial features indicated by dotted lines are determined based on the image of the user. One facial feature may be used as the facial characteristics, or several facial features may be used, or the whole face of the user may be used. In this example, the facial characteristics information is obtained from an image of the user. The image is obtained by the user taking a self-image which in this case is a two dimensional image but may be a three dimensional image. A three dimensional image may be obtained by moving an imaging device, such as that found in a mobile device, around the head and/or face of the user, and processing the image to determine the dimensions and/or the relative position of the features of the user. Alternatively, a three dimensional image may be obtained by a multi focus imaging device.

Additionally or alternatively, the information on the facial characteristics of the user may be obtained using the emitter and detector. Prior to use the user may perform a scanning motion of their face using the oral care device, where the oral care device is positioned at a predetermined distance from the face of the user. The received reflected energy may be used to collect information on the topology of the face of the user, for example, to create a three dimensional image. The facial characteristics information may be collected in real time, as the user uses the oral care device.

Additionally or alternatively, the facial characteristics information may include metadata such as the weight, height, complexion, gender and/or age of the user. This data may be collected by processing an image of the user or may be input by the user, using an application on a mobile phone or the like. The metadata may be used to estimate, or improve the estimation, of facial features of the user using a predetermined correlation between the size/position of facial features and metadata.

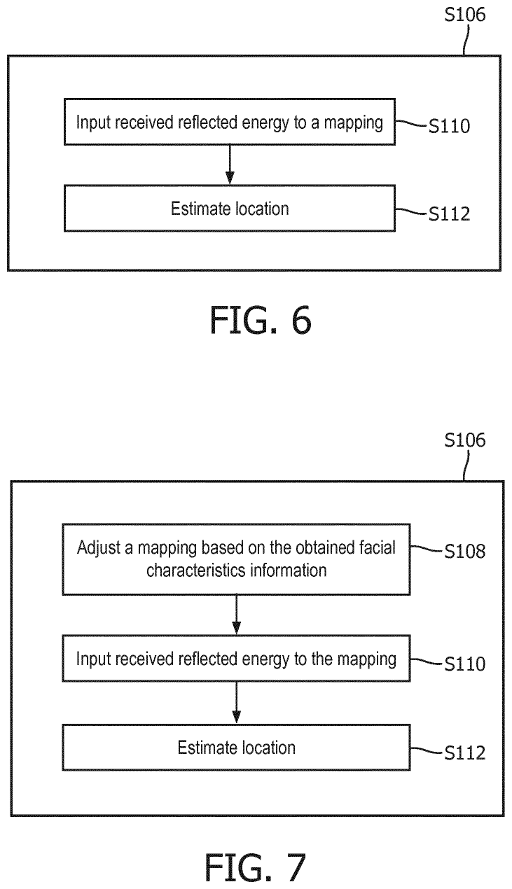

FIG. 6 shows an example of a method involved in step S106 of determining the position of the oral care device in the mouth of the user as shown in FIG. 4. Step S106 comprises the steps of S110, inputting the received reflected energy to the mapping, and thereby S112, estimating the location of the oral care device in the mouth of the user. The mapping is a trained (machine learning) algorithm which is developed during controlled, or guided, sessions with a diverse group of people, whereby the location of the oral care device in the mouth of a person is monitored while reflected energy is received. For example, the facial characteristics of each person of the group of people may be represented as a vector of parameters describing the surface of the face of a user which is used to train the algorithm. Thus, a general algorithm based on a generic person is provided which will estimate the location of an oral care device in the mouth of the user using the received reflected energy from the user.

FIG. 7 shows an example of the method as shown in FIG. 6, comprising the additional step S108, which defines adjusting a mapping based on the obtained facial characteristics information. Where the mapping is a machine learned algorithm, the user's facial characteristics are added as additional inputs to the algorithm, whereby the mapping is then adapted based on the user's facial characteristics. The facial characteristics of the user may be represented as a vector of parameters describing the surface of the face of a user, which may be fed as the additional input to the algorithm. The facial characteristics information is used to determine the location and dimensions of each facial feature of the user relative to one another. The location and dimensions of the facial features of the user are compared to the location and dimensions of the facial features upon which the mapping is based, and the mapping is adjusted so that when reflected energy from the face of the user is received, the mapping correlates the reflected energy with a facial feature of the user, rather than with a facial feature of the generic person. Thus, the location of the oral care device is indicated more accurately determined with respect to the mouth of the user.

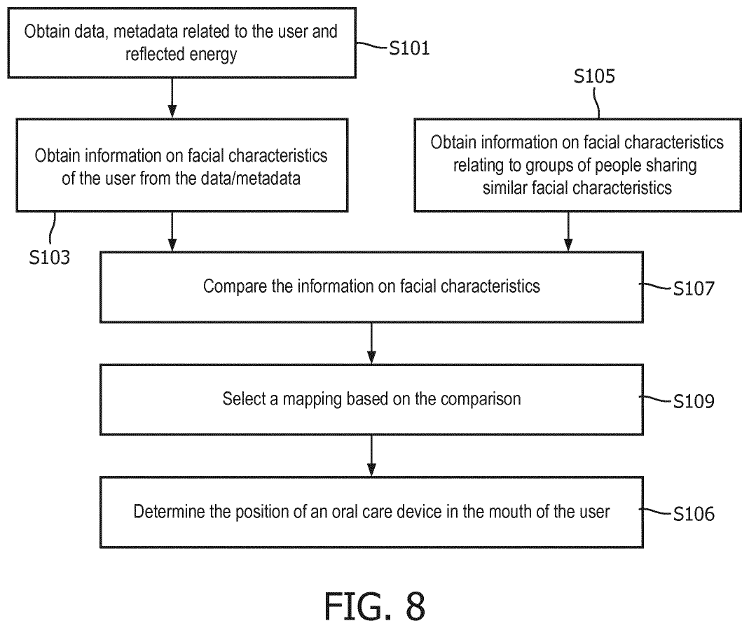

FIG. 8 shows an example of a method of monitoring the position of an oral care device in the mouth of the user which may be implemented alternatively or additionally to the method shown in FIG. 7. In FIG. 8, data and/or metadata related to the user and/or reflected energy are obtained, S101, and information on facial characteristics of the user are obtained or extracted from the data and/or metadata and/or reflected energy S103. Information on facial characteristics relating to groups of people sharing similar facial characteristics are obtained, S105. For example, the information may be obtained from a database which stores information on the facial characteristics of groups of people. The information on facial characteristics of the groups and the information on facial characteristics of the user are compared to determine which group of people has facial characteristics most similar to those of the user, S107. A mapping which corresponds to the determined group of people is then selected, S109. The selected mapping is an algorithm which has been trained using data compiled during a controlled brushing session, where the location of the oral care device relative to the facial characteristics of each member of the group of people is monitored while each member uses the oral cleaning device, such that reflected energy can be correlated to the location of the oral cleaning device. The reflected energy is then input to the selected mapping in order to determine the position of the oral care device in the mouth of the user S106. The process of step S106 may include the steps S108-S112 shown in FIG. 7.

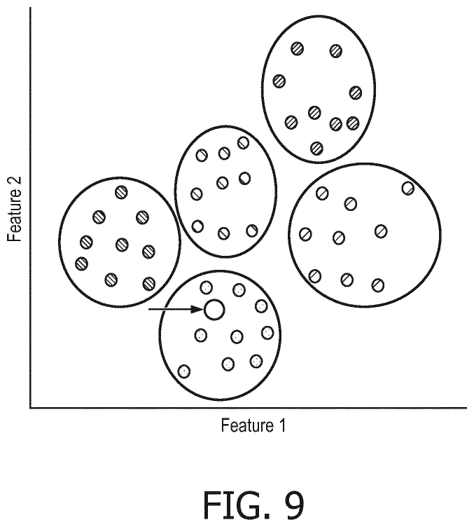

FIG. 9 shows an example of the grouping of people used to develop a plurality of mappings for different facial characteristics. In FIG. 9, a first feature, such as the distance of the nose from the mouth, is correlated with a second feature, such as the distance from the nose to the eyes. Each point on the graph indicates a different person. People with similar first and second features are grouped together, as indicated by the rings shown in FIG. 9. The data relating to people who are grouped together are used to develop a mapping which corresponds to that group of people. The point indicated by an arrow illustrates the user of the oral care device. The facial characteristics of the user are extracted using one of the abovementioned techniques. For example the first and second facial features described above are extracted. As is shown in this figure, the user has first and second facial features which are similar to a particular group of people, as they fall within the perimeter defined by a ring surrounding a particular group. The perimeter of the ring represents threshold values of the first and second characteristics. If the user falls within the threshold values of the first and second facial characteristics of a particular group of people, the user has facial characteristics most similar to those people. There may be provided any number of groups. The groups may comprise any number of people. Each group may only comprise one person, where the mapping corresponding to the person with the most similar facial features to the user is used as the selected mapping.

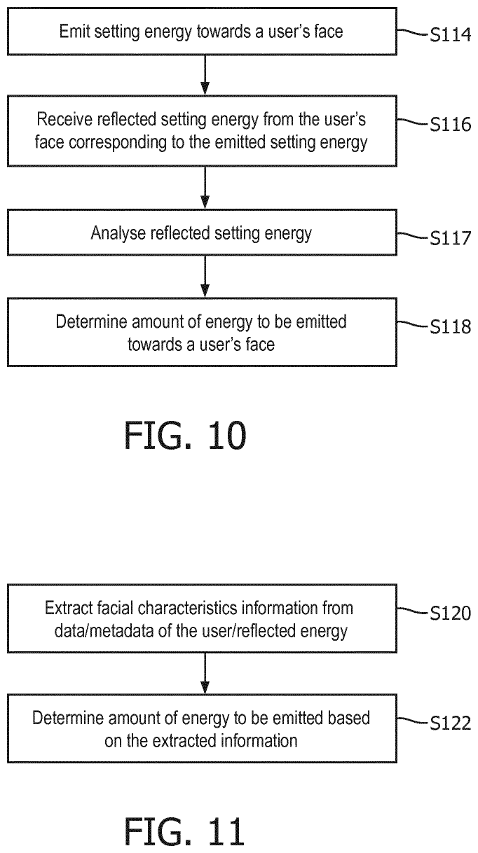

FIG. 10 shows an example of a method which may be applied additionally or alternatively to any of the previously specified methods. The method of FIG. 10 is performed before, for example, the step of emitting energy towards the user's face (step S100 in FIG. 4). At step S114, setting energy is emitted towards the user's face. This may be energy which is of a predefined intensity. The energy may be emitted from a predefined location, for example the oral care device may be held in front of a particular feature of the user, for example the nose, at a predefined distance. The setting energy may be emitted and reflected from the oral care device onto the particular feature of the user. The reflected setting energy corresponding to the emitted setting energy is received from the user's face S116. The reflected setting energy is then analysed S117, for example the amount or intensity of reflected setting energy which is received is compared to a predefined value of required energy. The amount of energy to be subsequently emitted towards the user's face is then determined, or corrected, based on the result of the comparison. For example, the amount of energy is increased or decreased based on the result of the comparison, so that the subsequently emitted energy returns a desired amount or intensity of reflected energy. Subsequently, any of the methods as described above may be implemented.

Alternatively or additionally, the method as set out in FIG. 11 may be implemented. At step S120, facial characteristics information may be extracted as is described above. At step S122, the amount of energy to be emitted may be determined based on the extracted information. For example, an image of the user may be analysed to determine the skin tone of the user. This may be used as the facial characteristics information to determine the amount of energy to be emitted in a step of emitting by increasing or decreasing the amount of energy by comparison of the skin tone to a predefined skin tone and energy.

While embodiments described herein include near infrared light energy sources and detectors, other types of energy may also be used. For example, alternative wavelengths of light, such as within the visible spectrum, radio frequency electromagnetic radiation forming a radar sensor, or electrostatic energy, such as in a mutual capacitance sensor may also be used. The sensor output may be derived from different aspects of the detected energy such as magnitude of the detected energy and/or phase or time delay between the energy source and the detected signal, time of flight.

It is understood that the embodiments of the present disclosure are not limited to the particular methodology, protocols, devices, apparatus, materials, applications, etc., described herein, as these may vary. It is also to be understood that the terminology used herein is used for the purpose of describing particular embodiments only, and is not intended to be limiting in scope of the embodiments as claimed. It must be noted that as used herein and in the appended claims, the singular forms “a,” “an,” and “the” include plural reference unless the context clearly dictates otherwise.

Unless defined otherwise, all technical and scientific terms used herein have the same meanings as commonly understood by one of ordinary skill in the art to which the embodiments of the present disclosure belong. Preferred methods, devices, and materials are described, although any methods and materials similar or equivalent to those described herein may be used in the practice or testing of the embodiments.

Although only a few exemplary embodiments have been described in detail above, those skilled in the art will readily appreciate that many modifications are possible in the exemplary embodiments without materially departing from the novel teachings and advantages of the embodiments of the present disclosure. The above-described embodiments of the present invention may advantageously be used independently of any other of the embodiments or in any feasible combination with one or more others of the embodiments.

Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

In addition, any reference signs placed in parentheses in one or more claims shall not be construed as limiting the claims. The word “comprising” and “comprises,” and the like, does not exclude the presence of elements or steps other than those listed in any claim or the specification as a whole. The singular reference of an element does not exclude the plural references of such elements and vice-versa. One or more of the embodiments may be implemented by means of hardware comprising several distinct elements. In a device or apparatus claim enumerating several means, several of these means may be embodied by one and the same item of hardware. The mere fact that certain measures are recited in mutually different dependent claims does not indicate that a combination of these measures may not be used to an advantage.

1. A method for monitoring the position of an oral care device in the mouth of a user, the method comprising:
emitting energy towards the user's face;
receiving reflected energy from the user's face corresponding to the emitted energy; and
determining the position of an oral care device in the mouth of the user using the received reflected energy and facial characteristics information of the user which relates to one or more facial features of the user.

2. The method as claimed in claim 1, wherein the facial characteristics information of the user further comprises at least one of: data relating to one or more facial characteristics of the user, metadata related to the user, facial characteristics information derived from the received reflected energy.

3. The method as claimed in claim 2, wherein the facial characteristics information of the user are at least one of: obtained from an image of the user, input by the user, obtained by processing the received reflected energy.

4. The method as claimed in claim 2, wherein the metadata is based on information on at least one of: the user's weight, height, complexion, gender, age.

5. The method as claimed in claim 1, wherein the position of the oral care device in the mouth of the user is determined using a mapping which indicates the position of the oral care device in the mouth of the user based on the received reflected energy and facial characteristics information of the user.

6. The method as claimed in claim 5, wherein the mapping is selected from a plurality of mappings, the selected mapping being a mapping which is determined to be the most relevant based on the facial characteristics information of the user.

7. The method as claimed in claim 6, wherein
each of the plurality of mappings relates to a different group of people, each group sharing at least one of: certain facial characteristics, metadata;
the at least one of: certain facial characteristics, metadata, of each group and the facial characteristics information of the user are used to identify which group is most relevant to the facial characteristics information of the user; and
the mapping corresponding to the identified group is selected.

8. The method as claimed in claim 5, wherein the mapping is adjusted based on the facial characteristics information.

9. The method as claimed in claim 1, wherein the method further comprises
emitting setting energy towards the user's face; and
receiving reflected setting energy from the user's face corresponding to the emitted energy; and
determining the amount of the energy to be emitted towards the user's face in the step of emitting energy based on the reflected setting energy; or
determining the amount of energy to be emitted towards the user's face in the step of emitting energy based on at least one of: data relating to one or more facial characteristics of the user, metadata related to the user.

10. The method as claimed in claim 1, wherein the energy is at least one of: electromagnetic energy, acoustic energy.

11. The method as claimed in claim 1, wherein the receiving of reflected energy comprises a measurement based on a measurement of at least one of: capacitance, reflected intensity, reflected polarization.

12. (canceled)

13. (canceled)

14. An oral care system comprising:
an oral care device having an energy emitter and an energy detector; and
a computing device configured to receive and process signals from energy emitted and received by the oral care device, and wherein the oral care system is configured to perform the method claimed in claim 1.

15. An oral care system as claimed in claim 14, wherein at least one of:
the oral care device (10) is a device chosen from a group of devices comprising: a toothbrush, a flossing device, an oral irrigator, a handle for receiving a care head for any of the foregoing devices, a care head for any of the foregoing devices; and
the computing device is comprised in at least one of: a remote server, an interface device to provide user information about the use of the oral care device; wherein the interface device is chosen from a group of interface devices comprising: a smart phone, a tablet, the oral care device, the care head.

16. An oral care device comprising an energy emitter and an energy detector, wherein the oral care device is configured to communicate with the computing device as claimed in claim 14.
